(19) United States
(12) Patent Application Publication
(10) Pub. No.: US 2024/0023945 A1
(43) Pub. Date: Jan. 25, 2024

(54) BALLOON-ANCHORED FLEXIBLE NEEDLE AND CATHETER FOR BIOPSY

(71) Applicants: NATIONAL UNIVERSITY HOSPITAL (SINGAPORE) PTL LTD, Singapore (SG); THE BIOFACTORY PTE LTD, Singapore (SG)

(72) Inventors: Kamarjit Singh MANGAT, Birmingham (GB); Gabriel Hong Chun TAN, Singapore (SG); Ronald Craig WIGHT, Singapore (SG); Chun Siong LEE, Singapore (SG)

(21) Appl. No.: 18/256,919

(22) PCT Filed: Dec. 10, 2021
(86) PCT No.: PCT/SG2021/050775

§ 371 Date: Jun. 10, 2023

(30) Foreign Application Priority Data
Dec. 10, 2020 (SG) 10202012399Y

(51) Int. Cl. A61B 10/04; A61M 25/10; A61M 25/00; A61B 10/02

(52) U.S. Cl. CPC A61B 10/04; A61M 25/10; A61M 25/0032; A61B 10/0275; A61B 2010/045

(57) ABSTRACT

The present disclosure provides a catheter for insertion into a subject and navigation to a target organ, the catheter comprising: a first elongated tube and a second elongated tube; the first elongated tube having a first lumen with a first proximal end and a first distal end; and the second elongated tube having a second lumen with a c-shaped cross section forming a channel with a second proximal end and a second distal end, the second elongated tube sharing a common wall with the first elongated tube.

### TECHNICAL FIELD

The present disclosure relates to biopsy devices, and more particularly to a biopsy device for acquiring a biopsy sample of a target organ. In particular, the present disclosure relates to a biopsy device with a flexible surgical instrument that can be inserted at a peripheral vascular access site through a catheter for performing soft tissue biopsy.

### BACKGROUND

Current methods for performing liver biopsies may include an inherent risk of severe complications, which may often result in patients opting to delay the biopsy procedure, thus delaying subsequent diagnosis and intervention of liver dysfunction. The methods may include open surgery, percutaneous liver biopsy (PLB), and trans-jugular liver biopsy (TJLB).

Open surgery liver biopsy is the direct removal of liver tissue during a laparoscopic, or surgical procedure. Open surgical liver biopsy in modern practice may be utilized when there is already a surgical procedure underway.

Percutaneous liver biopsy (PLB) may involve extracting a core sample of liver tissue using a biopsy needle inserted through the abdominal wall. In PLB, the liver capsule is punctured, and a high penetration depth is needed to reach the parenchyma. This procedure may often provide good biopsy samples, but the procedure is invasive, painful, and may carry a risk of significant complications, including a significant risk of death (1 in 250). If the first biopsy fails and additional biopsy samples are needed, additional needle punctures may be required, further increasing the risk of complications. Thus, PLB patients may be kept under observation for several hours after the procedure to ensure that there is no bleeding into the peritoneal cavity due to the puncturing of the liver capsule or vessels.

TJLB involves accessing the liver through the insertion of a stiff metal catheter into the right or left jugular vein and navigating the catheter through the right chamber of the heart and into the hepatic vein of the liver. A large bore needle directed down the catheter is used to core the liver tissue. Multiple samples are often needed for satisfactory analyses.

TJLB may avoid the risk of undetected bleeding into the peritoneum since any bleeding from the needle punctures as in PLB drains back into the hepatic vein. Since TJLB involves navigating a stiff metal catheter through major organs and blood vessels the procedures may result in significant complications such as hemorrhaging, arrhythmia, vessel perforation, pneumothorax, or death. Although TJLB may be considered to be safer than PLB, TJLB does incur new risks of complications related to its jugular access site.

Thus, there is a need for a transveous biopsy device that can be introduced into the peripheral venous system of the arm into the patient's body via the basilica/cephalic veins, for example, and flexibly navigated through the venous system for performing a soft tissue biopsy on a target organ, such as the liver, so as to reduce the risk of major complications associated with PLB and TJLB procedures.

International Patent Publication 2019/103694A1 by the current applicant and published May 31, 2019, the disclosure of which is incorporated herein by reference describes a balloon-anchored biopsy device. The balloon-anchored biopsy device disclosed had two elongated tubes with two lumens. However, although this was sufficient for most applications, there were some issues when the two elongated tubes were bent whilst navigating the vasculature resulting in kinking. This kinking causes the effective inner diameter of the tube to become an oval shape (i.e. same equivalent diameter) which then will clamp down on or excessively interfere with the advancement and firing of the biopsy needle. Furthermore, the angle at which the needle entered soft tissue was still not optimal.

It is thus desirable to provide an improved biopsy device given the current apparatus used for obtaining a core specimen from soft tissue.

### SUMMARY

According to embodiments of the present disclosure, there is provided a catheter for insertion into a subject and navigation to a target organ, the catheter comprising: a first elongated tube and a second elongated tube; the first elongated tube having a first lumen with a first proximal end and a first distal end; and the second elongated tube having a second lumen with a c-shaped cross section forming a channel with a second proximal end and a second distal end, the second elongated tube sharing a common wall with the first elongated tube.

According to some embodiments of the present disclosure, the first lumen is bean-shaped. Optionally, the common wall is located at a concave section of the bean-shaped lumen of the first elongated tube. Optionally, the first lumen has a closed curve shape with at least one indentation. Optionally, the common wall is located opposite to an opening of the channel of the second elongated tube. Optionally, the common wall has a reduced thickness compared to other walls of the catheter.

According to some embodiments, the catheter may further comprise an extension connected to the first distal end, the extension comprising a third proximal end and a third distal end, wherein the third proximal end is connected to the first distal end of the first elongated tube. Optionally, the extension is an extension of the common wall.

According to some embodiments, the catheter may further comprise a first tool for insertion into the first lumen of the first elongated tube. Optionally, the catheter may further comprise a second tool for insertion into the channel of the second elongated tube. Optionally, the second tool is a balloon catheter with a distal tip for insertion into the channel of the second elongated tube, wherein a section of the balloon catheter near the distal tip comprises a balloon that when inserted into a blood vessel of the target organ of the subject and inflated, anchors the extension in the blood vessel near a biopsy site in the target organ. Optionally, the first tool is a biopsy needle configured to exit the first distal end of the first lumen for penetration into tissue of the target organ at a predefined angle between a centre axis of the second tool and the extension and to acquire a biopsy sample of the target organ.

According to some embodiments, the extension further comprises a balloon wrap at the third distal end, the balloon wrap is configured to accept the balloon of the balloon catheter. Optionally, the balloon-wrap has a c-shaped cross section. Optionally, the extension comprises channel walls along its sides. Optionally, the channel walls decrease in height from the third proximal end to the third distal end.

According to some embodiments, the first proximal end of the first elongated tube and the second proximal end of the second elongated tube are coupled to a needle biopsy device. Optionally, the biopsy needle comprises a coring needle and a stylet needle. Optionally, the coring needle comprises one or more spiral cuts extending circumferentially around and longitudinally along the coring needle. Optionally, the one or more spiral cuts have a constant pitch. Alternatively, the one or more spiral cuts have a variable pitch.

According to some embodiments, the stylet needle comprises notches along its circumference longitudinally along the stylet needle. Optionally, the notches are under squared such that the notches are deeper than they are wider. Optionally, a base of the notches comprises one or more corner fillets. Optionally, the notches are diametrically opposed. Optionally, the notches are rotated 90 degrees axially.

According to some embodiments, the stylet needle comprises an indentation at a fourth distal end of the stylet needle. Optionally, the indentation has a flat base. Alternatively, the indentation has a concave base.

### BRIEF DESCRIPTION OF THE DRAWINGS

Features, aspects and advantages of the present disclosure will become better understood with regard to the following description and accompanying drawings in which:

FIG. 1A schematically illustrates a catheter body assembly for delivering a tool to a target organ in a subject, in accordance with some embodiments of the present disclosure;

FIG. 1B schematically illustrates a cross-section of a catheter, in accordance with some embodiments of the present disclosure;

FIG. 2 schematically illustrates a balloon catheter, in accordance with some embodiments of the present disclosure;

FIG. 3A schematically illustrates a top perspective view of an extension connected to a first distal end of a needle guide lumen at a distal exit of a catheter, in accordance with some embodiments of the present disclosure;

FIG. 3B schematically illustrates a top perspective view of an extension connected to a first distal end of a needle guide lumen at a distal exit of a catheter with a balloon catheter, in accordance with some embodiments of the present disclosure;

FIG. 3C schematically illustrates a bottom perspective view of an extension connected to a first distal end of a needle guide lumen at a distal exit of a catheter, in accordance with some embodiments of the present disclosure;

FIG. 3D schematically illustrates a bottom perspective view of an extension connected to a first distal end of a needle guide lumen at a distal exit of a catheter with a balloon catheter, in accordance with some embodiments of the present disclosure;

FIG. 4A schematically illustrates a distal end of a biopsy needle in a closed configuration, in accordance with some embodiments of the present disclosure;

FIG. 4B schematically illustrates a distal end of a biopsy needle in an open configuration, in accordance with some embodiments of the present disclosure;

FIG. 5A schematically illustrates a side view of a coring needle, in accordance with some embodiments of the present disclosure;

FIG. 5B schematically illustrates a bottom view of a coring needle, in accordance with some embodiments of the present disclosure;

FIG. 5C schematically illustrates a cross-section of a coring needle, in accordance with some embodiments of the present disclosure;

FIG. 5D schematically illustrates a distal end of a coring needle, in accordance with some embodiments of the present disclosure;

FIG. 6A schematically illustrates a top view of a stylet needle, in accordance with some embodiments of the present disclosure;

FIG. 6B schematically illustrates a side view of a stylet needle, in accordance with some embodiments of the present disclosure;

FIG. 6C schematically illustrates a blade of a stylet needle, in accordance with some embodiments of the present disclosure;

FIG. 6D schematically illustrates notches on a stylet needle, in accordance with some embodiments of the present disclosure;

FIG. 7A schematically illustrates a distal end of a catheter body assembly within a hepatic vein with an uninflated inflatable balloon, in accordance with some embodiments of the present disclosure; and

FIG. 7B schematically illustrates a distal end of a catheter body assembly within a hepatic vein of a liver with an inflated inflatable balloon and a biopsy needle penetrating a liver, in accordance with some embodiments of the present disclosure.

With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

Identical or duplicate or equivalent or similar structures, elements, or parts that appear in one or more drawings are generally labeled with the same reference numeral, optionally with an additional letter or letters to distinguish between similar entities or variants of entities, and may not be repeatedly labeled and/or described. References to previously presented elements are implied without necessarily further citing the drawing or description in which they appear.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

### DETAILED DESCRIPTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, use of the conjunction “or” as used herein is to be understood as inclusive (any or all of the stated options).

Embodiments of the present disclosure herein describe a core needle biopsy device for peripheral access (e.g., transcephalic venous access) that overcomes problems of insufficient force transfer from the handle to the cutting cannula and stylet at the biopsy site. The core needle biopsy device further includes safety mechanisms by preventing premature firing of the coring needle as well as providing separate locking mechanisms for the cannula and the biopsy needle.

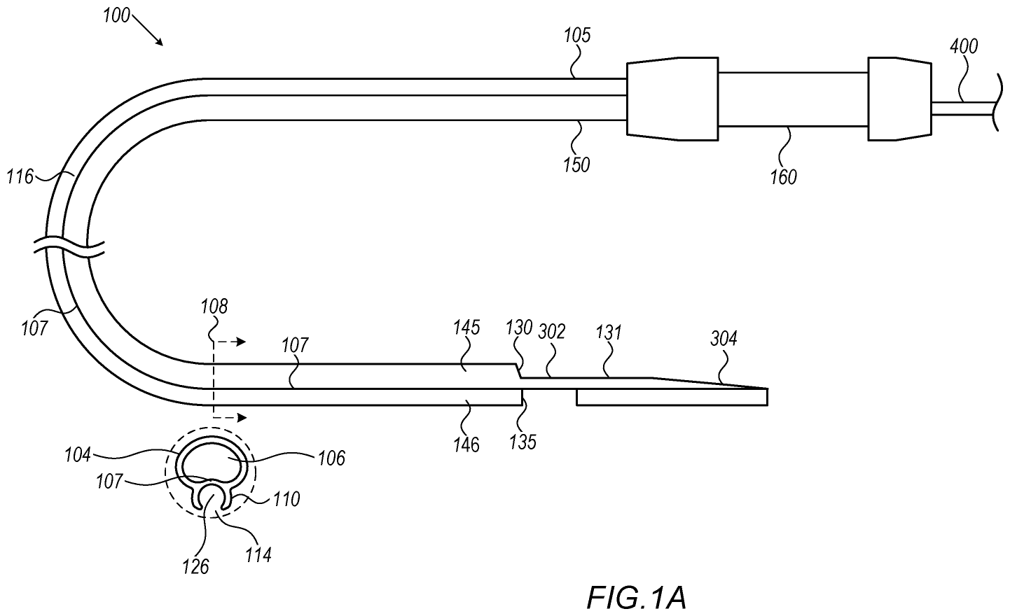

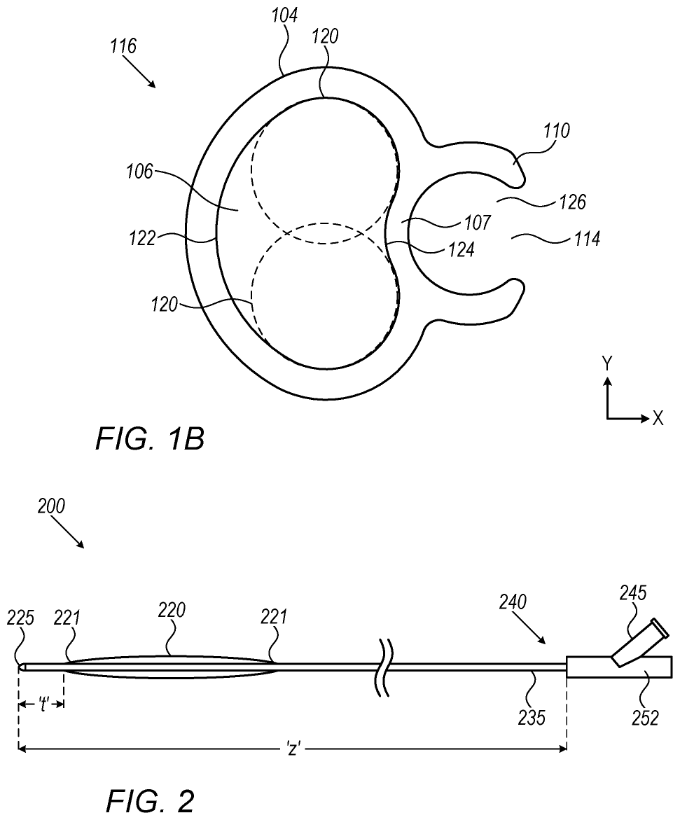

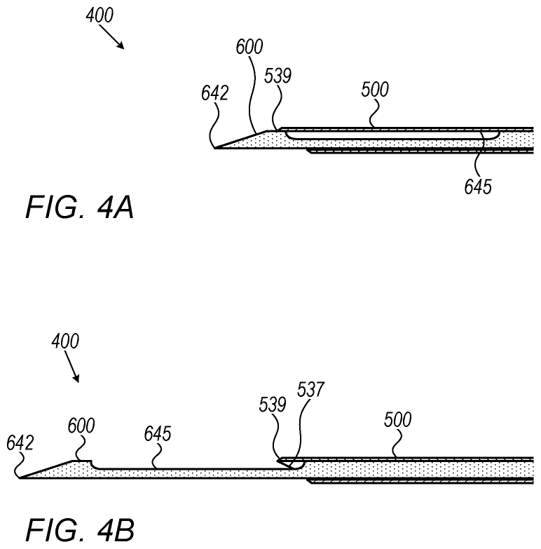

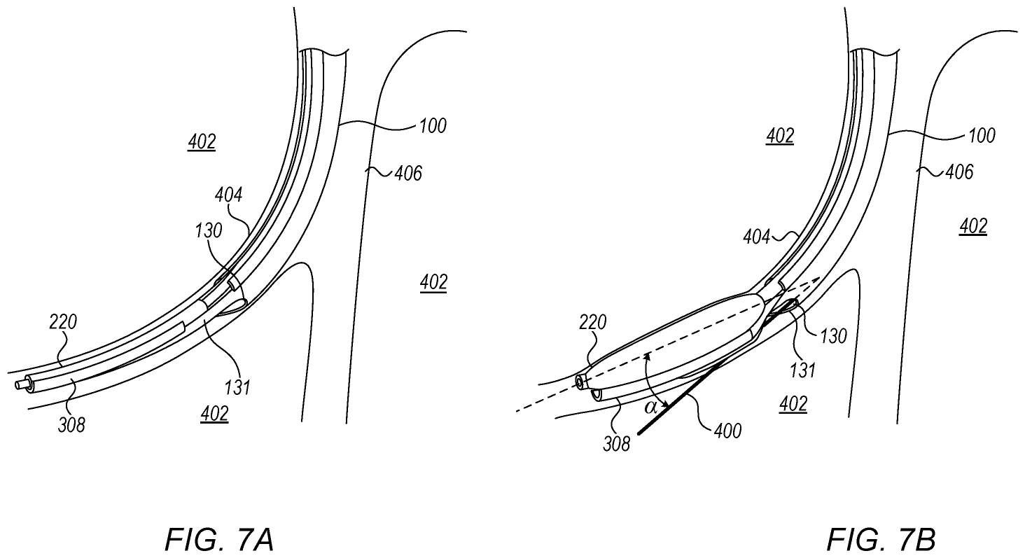

FIG. 1A schematically illustrates a catheter body assembly 100 for delivering a tool to a target organ in a subject, in accordance with some embodiments of the present disclosure. For the purpose of illustration, in the following description, the organ may be described as a liver, though it should be understood that the target organ may be any organ. In some embodiments, the catheter body assembly 100 is balloon-anchored. Catheter body assembly 100 comprises a catheter 116 and a ferrule 160. Catheter 116 may be formed from a medical grade polymer with 60 Shore D (plus minus 10). In accordance with some embodiments, catheter 116 comprises a first elongated tube and a second elongated tube. The first elongated tube comprises a first tool guide lumen 104 having a first proximal end 150, a first distal end 145 and a first distal exit 130, and the second elongated tube comprises a second tool lumen 110 having a second proximal end 105, a second distal end 146 and a second distal exist 135. In some embodiments of the disclosure, the catheter 116 is designed for delivering a first tool 400 to a target organ in a subject. The catheter 116 is further designed to couple a second tool 200 (see FIGS. 2, 3B, 3D, 7A and 7B) to be delivered to the organ in the subject. The catheter 116 may be used to deliver various surgical and non-surgical tools, such needles, balloons, clamps, suturing material, lighting devices, imaging devices, cutting and/or shearing devices, and the like. For the purpose of illustration, in the following description, the first tool 400 will be shown and described as a biopsy needle 400 (see FIGS. 4A, 4B, 7A and 7B) and the second tool 200 as a balloon catheter 200 (see FIGS. 2, 3B, 3D, 7A and 7B), though it should be understood that various other tools can be used in conjunction with the catheter 116. Similarly, the first tool guide lumen 104 will be shown and described as a needle guide lumen 104 and the second tool lumen 110 as a balloon catheter lumen 110.

In some embodiments of the present disclosure, the needle guide lumen 104 is configured as a bean-shaped lumen 106 adapted to guide biopsy needle 400 from the first proximal end 150 of needle guide lumen 104 to the first distal end 145 of needle guide lumen 104 and out of the first distal exit 130. In some embodiments, the balloon catheter lumen 110 has a c-shaped cross-section with an opening 114 extending the entire length of balloon catheter lumen 110, effectively forming a channel 126. In other embodiments, balloon catheter lumen 110 may be an enclosed lumen. Channel 126 may be adapted to couple or receive the balloon catheter 200, the balloon catheter 200 aligned longitudinally within the channel 126. Once coupled, the balloon catheter 200 may be secured by application of an adhesive, heat shrinking balloon catheter lumen 110, or selective localised reforming of balloon catheter lumen 110. This is to further prevent balloon catheter 200 from dislodging during tracking and navigation in a venous system or when stiff biopsy needle 400 is inserted.

In some embodiments of the present disclosure, bean-shaped lumen 106 of needle guide lumen 104 and c-shaped balloon catheter lumen 110 share a common wall 107. A cross-sectional cut 108 at catheter 116 schematically illustrates that bean-shaped lumen 106 of needle guide lumen 104 and c-shaped balloon catheter lumen 110 share a common wall 107. In some embodiments of the present disclosure, the cross-sectional wall thickness of catheter 116 is consistent. Preferably, opening 114 of balloon catheter lumen 110 is diametrically opposed to common wall 107 of needle guide lumen 104 and balloon catheter lumen 110 such that the cross-section of catheter 116 has a single section axis of symmetry.

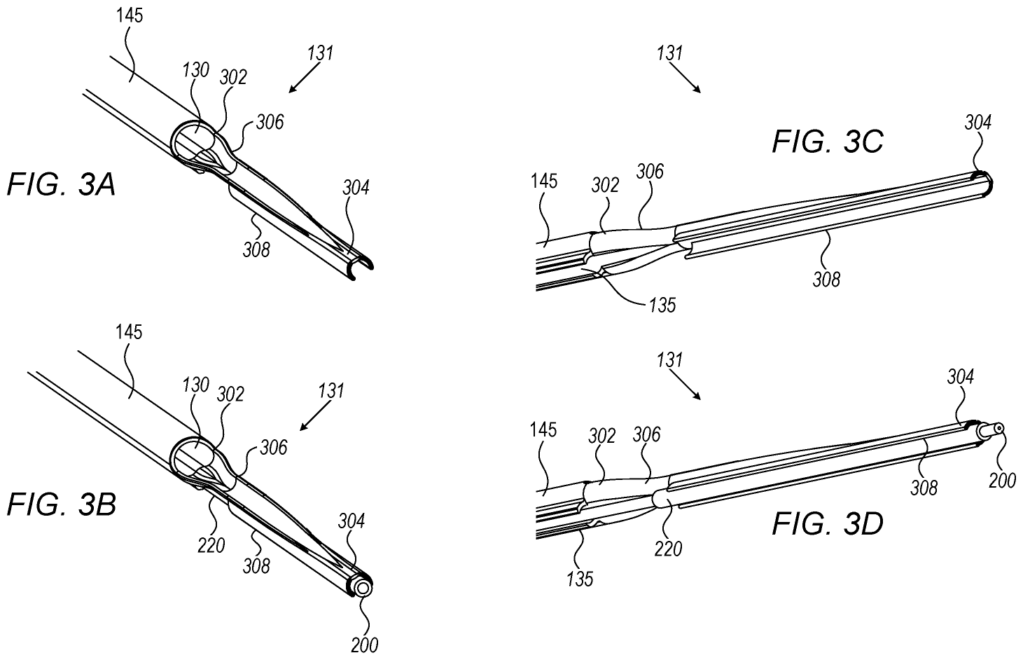

In some embodiments of the present disclosure, the first distal end 145 of needle guide lumen 104 may be terminated with a first distal exit 130. First distal end 145 may be connected to an extension 131 comprising a third proximal end 302 and a third distal end 304, the third proximal end 302 connected to the first distal end 145 of needle guide lumen 104. Extension 131 may be an extension of common wall 107. Extension 131 is adapted to guide biopsy needle 400 when it exits the first distal end 145 of needle guide lumen 104 through first distal exit 130 and into the liver parenchyma at a biopsy site. Extension 131 also prevents biopsy needle 400 from puncturing inflatable balloon 220 of balloon catheter 200 (see FIGS. 2, 3B, 3D, 7A and 7B) when it is inserted and inflated. The structure of extension 131 is further elaborated in relation to FIGS. 3A to 3D.

In some embodiments of the present disclosure, the first proximal end 150 of needle guide lumen 104 and the second proximal end 105 of balloon catheter lumen 110 may be connected to a ferrule 160. Ferrule 160 may incorporate a Tuohy-Borst adaptor to allow biopsy needle 400 or any surgical apparatus to be inserted. Ferrule 160 has an annulus of reduced diameter to accommodate a link that snaps on to ferrule 160 and is able to rotate about the annulus. This link may be rigidly attached to a needle biopsy device and anchor catheter 116 to the needle biopsy device. This coupling ensures a fixed relationship between the biopsy needle device and catheter 116 when biopsy needle 400 is inserted into the target organ.

FIG. 1B schematically illustrates a cross-section of a catheter 116, in accordance with some embodiments of the present disclosure. Catheter 116 has bean-shaped lumen 106 for a biopsy needle 400 to pass through and channel 126 for coupling or receiving a balloon catheter 200.

In some embodiments of the present disclosure, bean-shaped lumen 106 has a closed curve shape with at least one indentation. In some embodiments, the bean-shaped lumen 106 is shaped such that the top and bottom are shaped along the circumference a circle 120 of diameter between 1 and 3 mm (9 to 20 French on the French catheter scale) at the top and at the bottom sides of the lumen 106, with the entire cross-section of catheter 116 fitting within an inscribed diameter of between 3 and 6.7 mm (9 French to 20 French on the French catheter scale), while forming a curve shape at the common wall 107. The person skilled in the art would appreciate that the relative diameter of circle 120 of bean-shaped lumen 106 and overall diameter of catheter 116 may be adjusted to fit surgical instruments or balloon catheters of various sizes. For example, to fit a 16G biopsy needle, the diameter of circle 120 may be 1.95 mm with the overall diameter of catheter 116 being 4.67 mm. Preferably, there is a displacement of 0.236 mm along the x-axis between the centre of circle 120 and the centroid of the entire cross-section of catheter 116. Preferably, common wall 107 comprises a point of minimum wall thickness to locally concentrate deformation. As described, when the catheter 116 navigates the bends of the vasculature, this localised deformation minimises the deformation of bean-shaped lumen 106. This prevents bean-shaped lumen 106 from collapsing inwards when catheter 116 is bent along an axis parallel to the y-axis along a bending diameter of 90 mm, which is the tightest bending diameter that catheter 116 is expected to navigate through. The bean-shaped profile of bean-shaped lumen 106 allows catheter 116 to bend relatively easier in axes parallel to the Y-axis compared to axes parallel to the X-axis, thus allowing catheter 116 to orientate naturally to an arm entry to inferior vena cava to liver loop. The user will thus have clear tactile feedback of the position of extension 131 with respect to the liver orientation.

In some embodiments of the present disclosure, central region of bean-shaped lumen 106 has a concave arc 124 on one side and a convex arc 122 on the other side, both arcs diametrically opposed to each other. Preferably, the points of intersection of circles 120 are along the same plane as midpoint of concave arc 124 and convex arc 122. Preferably, central region of bean-shaped lumen 106 has a width of between 1 and 3 mm, and ideally 2.3 mm, from a midpoint of concave arc 124 to a midpoint of convex arc 122. A person skilled in the art would appreciate that this width may be sized to allow free movement of biopsy needle 400 through bean-shaped lumen 106.

In some embodiments of the present disclosure, balloon catheter lumen 110 has an overall diameter of between 1 and 4 mm, and ideally 2.47 mm. Preferably, balloon catheter lumen 110 has an inner diameter of between 0.5 and 2.5 mm, and ideally 1.67 mm forming channel 126. Preferably, there is a distance of 1.689 mm from the centroid of the cross-section of catheter 116 to the centre of the inner diameter of catheter guide 110. Preferably, balloon catheter lumen 110 has an opening 114 of between 0.3 and 2.5 mm, and ideally 1.19 mm. A person skilled in the art would appreciate that all the dimensions of balloon catheter lumen 110 may be sized to allow free movement of balloon catheter 200 within channel 126.

In some embodiments of the present disclosure, the walls of catheter 116 have a uniform thickness, except common wall 107 which is thinner than the other walls of catheter 116 and minimally not less than 50% of the walls of catheter 116. Preferably, the walls of catheter 116 are between 0.3 mm and 0.6 mm, and ideally 0.4 mm. Preferably, common wall 107 has a thickness of between 0.15 and 0.3 mm, and ideally 0.3 mm. The common wall 107 is diametrically opposite opening 114 and on the axis of symmetry through the cross section 108 of the catheter 116 (see FIG. 1A).

FIG. 2 is a schematic illustration of a balloon catheter 200, in accordance with some embodiments of the present disclosure. Balloon catheter 200 includes inflatable balloon 220 with tapering portions 221 that connect to the balloon catheter 200. Preferably, tapering portions 221 have an angle of between 15 and 60 degrees, and ideally between 30 to 60 degrees. Inflatable balloon 220 may be placed at a distal end of balloon catheter 200 and positioned at a predefined distance ‘t’ from a distal tip 225 of balloon catheter 200. Preferably, predefined distance ‘t’ is between 1 and 10 mm and ideally 3 mm. Optionally, there may be more than one inflatable balloon 220.

In some embodiments of the present disclosure, inflatable balloon 220 may be fabricated with a semi-compliant construction with a length of between 2 to 8 cm, and ideally 4 to 6 cm. Preferably, inflatable balloon 220 may have a diameter of between 5 to 15 mm, and ideally 10 mm, when inflated. The person skilled in the art would appreciate that the maximum diameter of inflatable balloon 220 may be sized to be no larger than the target vessel diameter by a factor of 20%. A fourth proximal end 235 of balloon catheter 200 may include a hub 240, common to over-the-wire balloon catheters. The distance between tip 225 and hub 240 of balloon catheter 200 may be a length ‘z’. Preferably, length ‘z’ is between 500 and 1100 mm, and ideally 900 mm. Hub 240 may include an inflation port 245 and a guidewire inlet/exit port 252. Inflatable balloon 220 may be inflated by air coupled into inflation port 245. Any suitable air valve or stopper mechanism, for example, may be used to hold the air within inflatable balloon 220 to keep it inflated or opened to deflate inflatable balloon 220.

FIGS. 3A-3D schematically illustrate perspective views of an extension 131 connected to the first distal end 145 of needle guide lumen 104 at distal exit 130 of catheter 116, with or without balloon catheter 200 inserted, in accordance with some embodiments of the present disclosure. FIG. 3A schematically illustrates a top perspective view of extension 131 connected to the first distal end 145 of needle guide lumen 104 at distal exit 130 of catheter 116, FIG. 3B schematically illustrates a top perspective view of extension 131 connected to the first distal end 145 of needle guide lumen 104 at distal exit 130 of catheter 116 with balloon catheter 200, FIG. 3C schematically illustrates a bottom perspective view of extension 131 connected to the first distal end 145 of needle guide lumen 104 at distal exit 130 of catheter 116, and FIG. 3D schematically illustrates a bottom perspective view of extension 131 connected to the first distal end 145 of needle guide lumen 104 at distal exit 130 of catheter 116 with balloon catheter 200, in accordance with some embodiments of the present disclosure.

In some embodiments of the present disclosure, extension 131 has a length of between 10 to 150 mm, and ideally 70 mm, to run the entire length of inflatable balloon 220. Preferably, extension 131 has channel walls 306 along its sides. Channel walls 306 are highest at proximal end 302 and gradually decrease in height as they move longitudinally along extension 131 until they reach distal end 304. Preferably, channel walls 306 have a height of 0.9 mm at proximal end 302, tapering to 0 mm at distal end 304.

In some embodiments of the present disclosure, the third distal end 304 of extension 131 includes a c-shaped balloon wrap 308 on its underside. C-shaped balloon wrap 308 is adapted to accept balloon catheter 200 and to hold inflatable balloon 220 of balloon catheter 200. Preferably, c-shaped balloon wrap 308 has a cross-section of a semi-circle with the same diameter as channel 126. Preferably, c-shaped balloon wrap 308 is connected longitudinally to extension 131 along a midpoint of its arc. Preferably, c-shaped balloon wrap 308 has a length of 40 mm extending from the third distal end 304 of extension 131.

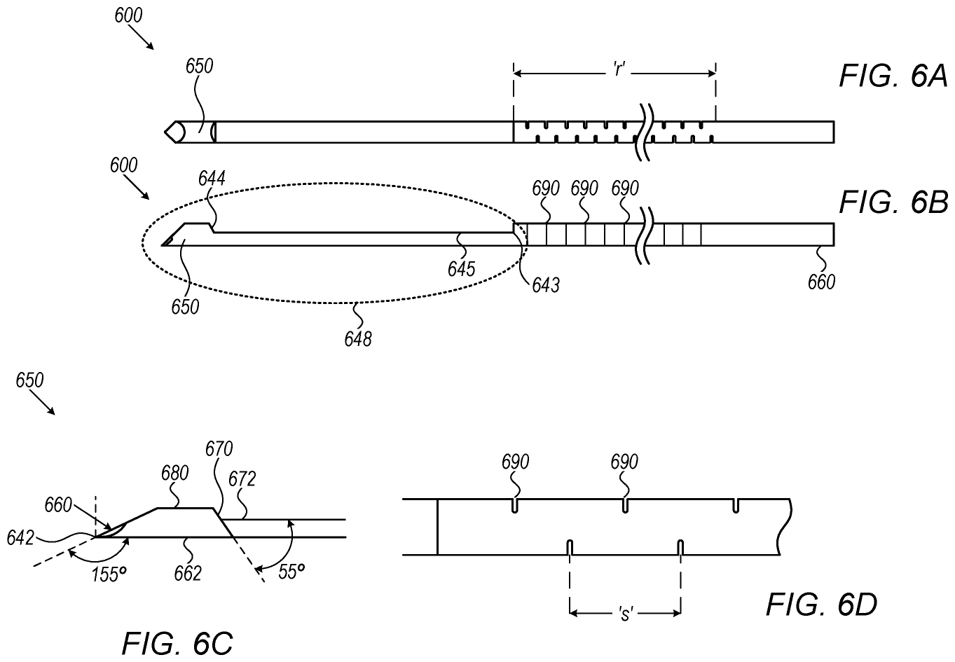

FIG. 4A schematically illustrates a distal end of a biopsy needle 400 in a closed configuration, while FIG. 4B schematically illustrates a distal end of a biopsy needle 400 in an open configuration, in accordance with some embodiments of the present disclosure. Biopsy needle 400 comprises a coring needle 500 and a stylet needle 600. Coring needle 500 is a thin-walled tubing that slides freely over stylet needle 600. Coring needle 500 has a cutting edge 537 with tip 539 at a distal end adapted to cut into biopsy tissue. Stylet needle 600 may be a 16g needle with a sample collection indentation 645 at its distal end 648 (see FIG. 6B) with a smaller cross-sectional area than the remainder of stylet needle 600. Stylet needle 600 may comprise a tip 642 at distal end 648 (see FIG. 6B). Coring needle 500 and stylet needle 600 may be made of any medical grade of stainless steel or titanium or metal alloy. Stylet needle 600 is inserted into coring needle 500. In a closed configuration (see FIG. 4A), sample collection indentation 645 forms a space between coring needle 500 and stylet needle 600 where a sample of biopsy tissue is collected. The tip 539 of cutting edge 537 of coring needle 500 and the tip 642 of stylet needle 600 are substantially opposite to one another. In an open configuration (see FIG. 4B), stylet needle 435 is advanced in relation to coring needle 500 and into biopsy tissue.

When coring needle 500 is subsequently advanced and biopsy needle 400 becomes a closed configuration, cutting edge 537 shears biopsy tissue and a sample is collected within sample collection indentation 645. The stylet needle 600 or entire biopsy needle 400 may be rotated within bean-shaped lumen 106 such that the sharp tip 642 is oriented away the extension 131, preventing the sharp tip 642 from puncturing that feature.

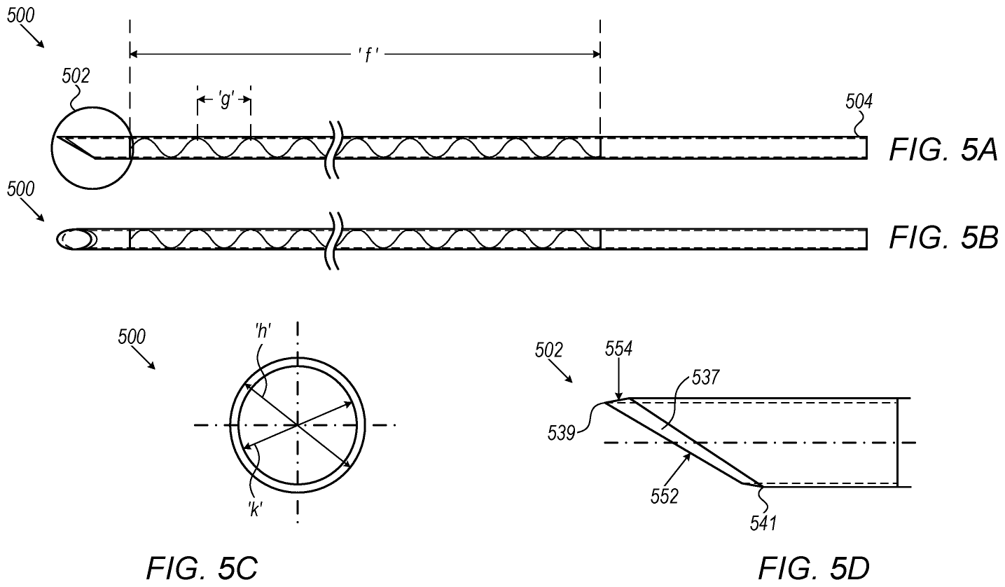

FIGS. 5A to 5D schematically illustrate coring needle 500, in accordance with some embodiments of the present disclosure. FIG. 5A schematically illustrates a side view of coring needle 500, FIG. 5B schematically illustrates a bottom view of coring needle 500, FIG. 5C schematically illustrates a cross-section of coring needle 500, and FIG. 5D schematically illustrates a distal end 502 of coring needle 500, in accordance with some embodiments of the present disclosure. Coring needle 500 comprises a distal end 502 and a proximal end 504. Preferably, coring needle 500 is of a length between 500 and 1000 mm, and ideally 918 mm. Proximal end 504 may be connected to a biopsy needle handle. Distal end 502 may comprise a cutting edge 537 with tip 539 and base 541 (see FIG. 5D).

In some embodiments of the present disclosure, coring needle 500 is adapted to be more flexible by the controlled removal of material from coring needle 500 such as introducing a spiral cut to a length ‘f’ along its axial length, with a pitch length ‘g’ between each spiral. Preferably, the width of the spiral cut may between 0.01 to 0.3 mm and ideally 0.1 mm. Optionally, the width of the spiral cut may gradually increase between distal end 502 to proximal end 504 of coring needle 500. Optionally, the width of the spiral cut may be 0.03 mm at distal end 502 and gradually increase to a maximum of 0.3 mm at proximal end 504 to allow maximum flexibility with minimal loss of push force. The person skilled in the art would appreciate that the adding of a spiral cut to the coring needle does not only make coring needle 500 more flexible, it also makes coring needle 500 behave more like a spring. By controlling the width of the spiral cut, local flexibility of the coring needle can be programmed. Preferably, the start of the spiral cut at the distal end 502 of the coring needle 500 is located 2.5 mm away from base 541 of cutting edge 537 at a distal end 502 of coring needle 500. Preferably, the end of the spiral cut at the proximal end 504 of coring needle 500 is located 20 mm away from proximal end 504 of coring needle 500. The length ‘f’ of the spiral cut may be any length extending along the coring needle 500. Optionally, there may be multiple spans of spiral cuts with same or differing lengths along length ‘f’ extending along coring needle 500, with solid continuous sections between spiral cuts and with spiral cut being between 1 to 50 mm depending on the length of coring needle 500 and flexibility to be achieved. The pitch length ‘g’ between each spiral may be of any length. Pitch length ‘g’ between each spiral may be constant or may vary along the entire length ‘f’ of the spiral cut. Preferably, pitch length ‘g’ between each spiral may be shorter at distal end 502 and longer at proximal end 504. Preferably, pitch length ‘g’ is between 2 to 10 mm, and ideally 6 mm. Optionally, coring needle 500 may have different discrete sections with different pitches that coincide with the different bends of the vasculature. At the most distal end 502 of the coring needle 500, the pitch length may be the shortest, for instance 2 mm, to provide the most flexible tip to navigate the most number of bends. The second and more proximal discrete section may have pitch length of 4 mm to balance the flexibility of coring needle 500 and the ability to transfer firing force from the needle biopsy device. The third and most proximal discrete section proximate to proximal end 504 of coring needle 500 may have the longest pitch length, for example 6 mm, since it hardly navigates the vasculature. Each section may be between 10 and 500 mm in length depending on the anticipated geometry of the vasculature that must be navigated. Between each discrete section, there may be sections of 0.1 mm to 5 mm of the coring needle 500 that do not have any spiral cut. Optionally, the pitch length may vary continuous throughout the coring needle 500 from 2 mm at the most distal end to 6 mm at the most proximal end. The person skilled in the art would appreciate that the pitch ‘g’ of the coring needle 500 must be sufficiently small such that when the coring needle 500 is guided through needle guide lumen 104 in the vicinity of the brachiocephalic vein, each spiral can adapt to the radius of the brachiocephalic vein without each individual spiral segment adopting a linear orientation. If the spiral segments adopt a linear orientation, the edge of each spiral would be misaligned and the relatively square corner of each edge could potentially abrade the inside of needle guide lumen 104. A person skilled in the art would also appreciate that if the pitch ‘g’ is too small, the sum of all the gaps between each spiral would compress during firing, effectively reducing the penetration depth of the coring needle 500 although this expected reduction in penetration depth can be compensated by pre-adjusting the stroke of the coring needle 500 firing mechanism.

In some embodiments of the present disclosure, coring needle 500 may have an outer diameter of ‘h’ and an inner diameter of ‘k’ (see FIG. 5C). Preferably, diameter ‘h’ is between 1 and 2 mm, and ideally 1.65 mm. Preferably, diameter ‘k’ is between 1 and 2 mm, and ideally 1.47 mm.

In some embodiments of the present disclosure, cutting edge 537 at distal end 502 of coring needle 500 may have a tip 539 and base 541 (see FIG. 5D). In some embodiments of the present disclosure, cutting edge 537 is continuous acute-angled face along the circumference of the distal end 502. In some embodiments, cutting edge 537 may have a Menghini point of length between 2 and 4 mm, and ideally 2.96 mm, with primary bevel 552 between 15 and 60 degrees, and ideally 30 degrees, and secondary bevel 554 between 5 and 30 degrees, and ideally 12 degrees.

FIGS. 6A to 6D schematically illustrate stylet needle 600, in accordance with some embodiments of the present disclosure. FIG. 6A schematically illustrates a top view of stylet needle 600, FIG. 6B schematically illustrates a side view of stylet needle 600, FIG. 6C schematically illustrates a blade 650 of stylet needle 600, and FIG. 6D schematically illustrates notches on stylet needle 600, in accordance with some embodiments of the present disclosure. Stylet needle 600 comprises fourth distal end 648 and fourth proximal end 660. Proximal end 660 may be connected to a biopsy needle handle. Fourth distal end 648 comprises a blade 650 and a sample collection indentation 645. Sample collection indentation 645 has a distal end 644 and a proximal end 643. Preferably, stylet needle 600 has a diameter of between 1 to 2 mm, and ideally 1.37 mm. Preferably, sample collection indentation 645 has a depth of between 0.4 to 0.6 mm, and ideally 0.55 mm. The person skilled in the art would appreciate that the depth of sample collection indentation should not be more than half of the cross-sectional area of stylet needle 600. Sample collection indentation 645 may have a surface, between distal end 644 and proximal end 643, that is flat or curved (concave). A concave surface can allow for the collection of an increased volume of sample while retaining similar stylet needle 600 shaft stiffness. Adequate shaft stiffness is required such that the stylet needle 600 does not buckle or bend significantly when puncturing the organ tissue.

In some embodiments of the present disclosure, blade 650 has a leading edge 660, a trailing edge 670, a level surface 680 and a base 662. Flat surface 680 joins leading edge 660 and trailing edge 670. Base 662 is joined to leading edge 660 at tip 642. Preferably, leading edge 660 has a perpendicular length from tip 642 of between 1 and 4 mm, and ideally 2.9 mm. Preferably, leading edge 660 has an angle of between 110 and 170 degrees, and ideally 155 degrees, from a base 662 of blade 650. Trailing edge 670 joins level surface 680 and sample collection indentation 645. Trailing edge 670 meets proximal end 644 of sample collection indentation at point 672. Preferably, trailing edge 670 has an angle of between 90 and 165 degrees, and ideally 125 degrees, from sample collection indentation 645. Preferably, level surface 680 has a perpendicular length of between 0.5 mm and 5 mm, and ideally 3 mm, from point 672.

In some embodiments of the present disclosure, stylet needle 600 is adapted to be more flexible by introducing notches along a length ‘r’ along its axial length, with a pitch length ‘s’ between each notch. Because the sample collection indentation 645 presents a smaller cross-sectional area than the remainder of the stylet needle 600, the proximal end 643 of the sample collection indentation 645, in relation to the distal end 648 that penetrates the parenchyma, is representative of the fixed end of a cantilever such that for any force applied to the distal end 648 of the stylet needle 600, the maximum bending moment, and stress concentration, will occur at said proximal end 643 of the sample collection indentation 645 due to the abrupt change of cross sectional area. Thus, notches 690 are introduced to the stylet needle 600 to reduce the tendency for the maximum bending moment in said stylet needle 600 to occur at the proximal end 643 of the sample collection indentation 645. The notches 690 enable stylet needle 600 to adopt a progressive rate of change of curvature within the more flexible spiral cut coring needle 500 as both negotiate the needle guide lumen 104 as the forces of bending are effectively distributed along stylet needle 600 instead of being concentrated at the proximal end 643 of the sample collection indentation 645, with each notch itself becoming a stress concentrator. The person skilled in the art would thus appreciate that the length ‘r’ and pitch ‘s’ of coring needle 500 are tuned to stylet needle 600 for the various gauges. Preferably, pitch ‘s’ is between 0.5 and 5 mm and ideally 2 mm. The length ‘r’ can be any length extending along the stylet needle 600. Preferably, notches 690 have a width of between 0.01 and 0.3 mm, and ideally 0.1 mm. Preferably, notches 690 have a depth of between 0.1 and 0.55 mm, and ideally 0.2 mm deep. Preferably, notches 690 have a depth of not more than 30% of the diameter of stylet needle 600 and are not deeper than the thinnest point on the sample collection indentation 645 so as not to compromise the overall strength of the stylet needle 600. Preferably, notches 690 are deeper than they are wider to give a profile of an under square to minimise interference with the spiral cut patterns. For example, notches 690 may have a depth of 0.2 mm and a width of 0.1 mm. Preferably, base of notches 690 have corner fillets to reduce the stress concentration at the inside corner of a right-angle joint. Preferably, notches 690 may be orientated at 90 degrees offset to each other, with the first notch 690 proximate to sample collection indentation 645 orientated parallel to sample collection indentation 645. It can be appreciated that the edges of the notches 690 of the stylet needle 600 are aligned to not interfere with the spiral cut patterns of the coring needle 500. This prevents any snagging of the two slit surfaces when they move past each other.

FIG. 7A schematically illustrates a distal end of catheter body assembly 100 within a hepatic vein 404 with an uninflated inflatable balloon 220, while FIG. 7B schematically illustrates a distal end of catheter body assembly 100 within a hepatic vein 404 with an inflated inflatable balloon 220 and a biopsy needle 400 penetrating liver 402, in accordance with some embodiments of the present disclosure. Distal end of catheter body assembly 100 may be navigated through the venous system to liver 402 through inferior vena cava 406 and into hepatic vein 404, such as the middle hepatic vein. Throughout navigation, biopsy needle 400 may be kept within catheter body assembly 100 without exiting distal exit 130.

In some embodiments of the present disclosure, once distal end of catheter body assembly 100 with balloon catheter 200 within c-shaped balloon wrap 308 is positioned at a hepatic vein 404, inflatable balloon 220 may be inflated to a diameter (e.g., up to 20% larger than the vein diameter) so as to anchor distal end of catheter body assembly 100 and extension 131 in hepatic vein 404. When inflatable balloon 220 is inflated, extension 131 and c-shaped balloon wrap 308 are pushed against vessel walls of hepatic vein 404. Since distal exit 130, from where biopsy needle 400 exits, is proximal to inflated balloon 220, biopsy needle 400 should be directed obliquely along extension 131 towards liver parenchyma 402. Biopsy needle 400 would thus advance into liver parenchyma 402 at an angle α between the centre axis of inflatable balloon 220 of balloon catheter 200 and the centre axis of extension 131 (see FIG. 7B). Before inflatable balloon 220 is inflated, angle α is 0 degrees. Angle α formed when inserting biopsy needle 400 into the liver 402 is dependent on the angle of tapering section 221 of inflatable balloon 220. The lower the angle of tapering section 221 of inflatable balloon, the lower the offset of extension 131. Biopsy needle 400 would be deflected to a lesser degree, lowering angle α of penetration. Thus, the angle α of insertion may be adapted to the human anatomy instead of being a predefined angle. Preferably, angle α is between 5 to 20 degrees, and preferably between 10 to 15 degrees. Channel walls 306 along extension 131 guide biopsy needle 400 such that biopsy needle 400 does not veer off to the left or right when advancing into the liver 402.

Different embodiments are disclosed herein. Features of certain embodiments may be combined with features of other embodiments; thus certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A catheter for insertion into a subject and navigation to a target organ, the catheter comprising:
a first elongated tube and a second elongated tube;
the first elongated tube having a first lumen with a first proximal end and a first distal end; and
the second elongated tube having a second lumen with a c-shaped cross section forming a channel with a second proximal end and a second distal end, the second elongated tube sharing a common wall with the first elongated tube.

2. The catheter according to claim 1, wherein the first lumen is a bean-shaped lumen.

3. The catheter according to claim 2, wherein the common wall is located at a concave section of the bean-shaped lumen of the first elongated tube.

4. The catheter according to claim 1, wherein the first lumen has a closed curve shape with at least one indentation.

5. The catheter according to claim 1, wherein the common wall is located opposite to an opening of the channel of the second elongated tube.

6. The catheter according to claim 1, wherein the common wall has a reduced thickness compared to other walls of the catheter.

7. The catheter according to claim 1, further comprising an extension connected to the first distal end, the extension comprising a third proximal end and a third distal end, wherein the third proximal end is connected to the first distal end of the first elongated tube.

8. The catheter according to claim 7, wherein the extension is an extension of the common wall.

9. The catheter according to claim 7, further comprising a first tool for insertion into the first lumen of the first elongated tube.

10. The catheter according to claim 9, further comprising a second tool for insertion into the channel of the second elongated tube.

11. The catheter according to claim 10, wherein the second tool is a balloon catheter with a distal tip for insertion into the channel of the second elongated tube, wherein a section of the balloon catheter near the distal tip comprises a balloon that when inserted into a blood vessel of the target organ of the subject and inflated, anchors the extension in the blood vessel near a biopsy site in the target organ.

12. The catheter according to claim 10, wherein the first tool is a biopsy needle configured to exit the first distal end of the first lumen for penetration into tissue of the target organ at a predefined angle between a centre axis of the second tool and the extension and to acquire a biopsy sample of the target organ.

13. The catheter according to claim 11, wherein the extension further comprises a balloon wrap at the third distal end, the balloon wrap is configured to accept the balloon of the balloon catheter.

14. The catheter according to claim 13, wherein the balloon wrap has a c-shaped cross section.

15. The catheter according to claim 7, wherein the extension comprises channel walls along its sides.

16. The catheter according to claim 15, wherein the channel walls decrease in height from the third proximal end to the third distal end.

17. The catheter according to claim 1, wherein the first proximal end of the first elongated tube and the second proximal end of the second elongated tube are coupled to a needle biopsy device.

18. The catheter according to claim 12, wherein the biopsy needle comprises a coring needle and a stylet needle.

19. The catheter according to claim 18, wherein the coring needle comprises one or more spiral cuts extending circumferentially around and longitudinally along the coring needle.

20. The catheter according to claim 19, wherein the one or more spiral cuts have a constant pitch.

21. The catheter according to claim 19, wherein the one or more spiral cuts have a variable pitch.

22. The catheter according to claim 18, wherein the stylet needle comprises notches along its circumference longitudinally along the stylet needle.

23. The catheter according to claim 22, wherein the notches are under squared such that the notches are deeper than they are wider.

24. The catheter according to claim 22, wherein a base of the notches comprises one or more corner fillets.

25. The catheter according to claim 22, wherein the notches are diametrically opposed.

26. The catheter according to claim 22, wherein the notches are rotated 90 degrees axially.

27. The catheter according to claim 18, wherein the stylet needle comprises an indentation at a fourth distal end of the stylet needle.

28. The catheter according to claim 27, wherein the indentation has a flat base.

29. The catheter according to claim 27, wherein the indentation has a concave base.
